(19) United States
(12) Patent Application Publication
(10) Pub. No.: US 2026/0334817 A1
(43) Pub. Date: Oct. 29, 2026

(54) GAIT ANALYSIS DEVICE, GAIT ANALYSIS SYSTEM, GAIT ANALYSIS METHOD, AND NON-TRANSITORY RECORDING MEDIUM

(71) Applicant: NEC Corporation, Tokyo (JP)

(72) Inventor: Hiroshi KAJITANI, Tokyo (JP)

(73) Assignee: NEC Corporation, Tokyo (JP)

(21) Appl. No.: 19/646,820

(22) Filed: Apr. 14, 2026

(30) Foreign Application Priority Data
Apr. 25, 2025 (JP) 2025-073164

(51) Int. Cl. A61B 5/11; A61B 5/00

(52) U.S. Cl. CPC A61B 5/112; A61B 5/0004; A61B 5/6823; A61B 5/6829

(57) ABSTRACT

A gait analysis device that includes, a reception unit that receives pieces of sensor data measured by a first measurement device arranged along a body axis of a user, a second measurement device arranged on a left foot of the user, and a third measurement device arranged on a right foot of the user, a synchronization processing unit that synchronizes the pieces of the sensor data respectively received from the first measurement device, the second measurement device, and the third measurement device with each other, a measurement unit that measures a double support time by using the sensor data received from the first measurement device, an information generation unit that generates gait information according to the double support time measured, and an output unit that outputs display information including the gait information generated.

This application is based upon and claims the benefit of priority from Japanese patent application No. 2025- 073164, filed on Apr. 25, 2025, the disclosure of which is incorporated herein in its entirety by reference.

### TECHNICAL FIELD

The present disclosure relates to a gait analysis device, a gait analysis system, a gait analysis method, and a program.

### BACKGROUND ART

With growing interest in healthcare, services that provide information according to gait have attracted attention. For example, a technique for analyzing gait by using sensor data measured by a sensor implemented on footwear such as shoes has been developed. In time-series data of the sensor data, a feature related to the physical condition appears. For example, a double support time (DST) indicating a time during which both feet are grounded during walking is an index of gait ability. The double support time is measured by use of an inflection point of time-series data of a roll angle included in the sensor data.

PTL 1 (JP 2024-140556 A) discloses a gait trajectory measuring device for the purpose of measuring a continuous gait trajectory. The device of PTL 1 includes a sensor and a processing device. The sensor is attached to a lower leg of a walker. The processing device calculates a gait trajectory, based on the time-series data including a three-dimensional acceleration and a three-dimensional angular velocity generated by the sensor. The processing device calculates gait trajectories for respective steps, based on the data generated by the sensor. The processing device calculates a rotation angle by integrating angles formed by adjacent stride vectors. The processing device generates a continuous gait trajectory by connecting together the gait trajectories for respective steps by using the rotation angle.

In the method of PTL 1, a gait trajectory is calculated by use of acceleration and angular velocity data measured by the sensor attached to the lower leg. In the method of PTL 1, since only the movement of the lower leg is detected, a grounded state of a foot portion has not been able to be accurately grasped. In addition, in the method of PTL 1, pieces of sensor data measured by respective sensors attached to both legs have not been able to be accurately synchronized with each other. For that reason, in the method of PTL 1, the double support time has not been able to be accurately measured.

An object of the present disclosure is to provide a gait analysis device, a gait analysis system, a gait analysis method, and a program capable of accurately measuring a double support time by synchronizing pieces of sensor data measured by a plurality of measurement devices with each other.

### SUMMARY

A gait analysis device according to an aspect of the present disclosure includes, a reception unit that receives pieces of sensor data measured by a first measurement device arranged along a body axis of a user, a second measurement device arranged on a left foot of the user, and a third measurement device arranged on a right foot of the user, a synchronization processing unit that synchronizes the pieces of the sensor data respectively received from the first measurement device, the second measurement device, and the third measurement device with each other, a measurement unit that measures a double support time by using the sensor data received from the first measurement device, an information generation unit that generates gait information according to the double support time measured, and an output unit that outputs display information including the gait information generated.

In a gait analysis method according to an aspect of the present disclosure, a computer receives pieces of sensor data measured by a second measurement device arranged on a left foot of a user, a third measurement device arranged on a right foot of the user, and a first measurement device arranged along a body axis of the user, synchronizes the pieces of the sensor data respectively received from the first measurement device, the second measurement device, and the third measurement device with each other, measures a double support time by using the sensor data received from the first measurement device, generates gait information according to the double support time measured, and outputs display information including the gait information generated.

A program according to an aspect of the present disclosure causes a computer to execute, processing of receiving pieces of sensor data measured by a second measurement device arranged on a left foot of a user, a third measurement device arranged on a right foot of the user, and a first measurement device arranged along a body axis of the user, processing of synchronizing the pieces of the sensor data respectively received from the first measurement device, the second measurement device, and the third measurement device with each other, processing of measuring a double support time by using the sensor data received from the first measurement device, processing of generating gait information according to the double support time measured, and processing of outputting display information including the gait information generated.

According to the present disclosure, it is possible to provide a gait analysis device, a gait analysis system, a gait analysis method, and a program capable of accurately measuring a double support time by synchronizing pieces of sensor data measured by a plurality of measurement devices with each other.

### BRIEF DESCRIPTION OF THE DRAWINGS

FIG. 1 is a block diagram illustrating a configuration of a gait analysis system in the present disclosure;

FIG. 2 is a conceptual diagram illustrating an arrangement example of a measurement device included in the gait analysis system in the present disclosure;

FIG. 3 is a conceptual diagram illustrating an example of a coordinate system set in the measurement device of the present disclosure;

FIG. 4 is a conceptual diagram illustrating an arrangement example of the measurement device in the present disclosure;

FIG. 5 is a conceptual diagram illustrating an arrangement example of the measurement device in the present disclosure;

FIG. 6 is a block diagram illustrating an example of a configuration of the measurement device in the present disclosure;

FIG. 7 is a conceptual diagram for describing a human body plane set for a human body;

FIG. 8 is a block diagram illustrating an example of a configuration of a gait analysis device in the present disclosure;

FIG. 9 is a conceptual diagram for describing a change in the direction of the measurement device in the present disclosure;

FIG. 10 is a conceptual diagram for describing a change in the direction of the measurement device in the present disclosure;

FIG. 11 is a graph illustrating an example of sensor data measured by the measurement device of the present disclosure;

FIG. 12 is a graph illustrating an example of sensor data measured by the measurement device of the present disclosure;

FIG. 13 is a graph illustrating an example of sensor data measured by the measurement device of the present disclosure;

FIG. 14 is a graph illustrating an example of sensor data measured by the measurement device of the present disclosure;

FIG. 15 is a graph illustrating an example of sensor data measured by the measurement device of the present disclosure;

FIG. 16 is a conceptual diagram illustrating a display example of gait information output from the gait analysis device in the present disclosure;

FIG. 17 is a conceptual diagram illustrating a display example of gait information output from the gait analysis device in the present disclosure;

FIG. 18 is a conceptual diagram illustrating a display example of gait information output from the gait analysis device in the present disclosure;

FIG. 19 is a flowchart illustrating an example of an operation of the gait analysis device in the present disclosure;

FIG. 20 is a block diagram illustrating an example of a configuration of a gait analysis device in the present disclosure;

FIG. 21 is a flowchart illustrating an example of an operation of the gait analysis device in the present disclosure; and

FIG. 22 is a block diagram illustrating an example of a hardware configuration that executes control and processing in the present disclosure.

### EXAMPLE EMBODIMENT

Hereinafter, modes for carrying out the present disclosure will be described with reference to the drawings. In the present disclosure, the drawings used in description of each example embodiment are associated with one or more example embodiments. Elements included in each drawing may apply to one or more example embodiments. The example embodiments described below have technically preferable limitations for carrying out the present disclosure, but the scope of the disclosure is not limited to the following. All the drawings used in the following description of the example embodiments indicate similar parts with the same reference signs unless otherwise specified. In the following example embodiments, repeated description of similar configurations and operations may be omitted in some cases. The directions of the arrows in the drawings indicate examples of flows of signals, data, and the like and do not limit the flows of signals, data, and the like.

### First Example Embodiment

First, a gait analysis system according to a first example embodiment will be described with reference to the drawings. The gait analysis system of the present example embodiment includes a plurality of measurement devices each including an acceleration sensor and an angular velocity sensor. The measurement device is arranged on footwear of a user. In addition, the measurement device is arranged at a part along the body axis such as the waist. The gait analysis system of the present example embodiment synchronizes pieces of sensor data measured by the plurality of measurement devices with each other. The gait analysis system of the present example embodiment measures a double support time (DST) by using the synchronized pieces of the sensor data. The double support time indicates a time during which both feet are grounded during walking. The double support time is an index of gait ability. The gait analysis system of the present example embodiment generates information regarding the gait ability of the user according to the measured double support time.

### Configuration

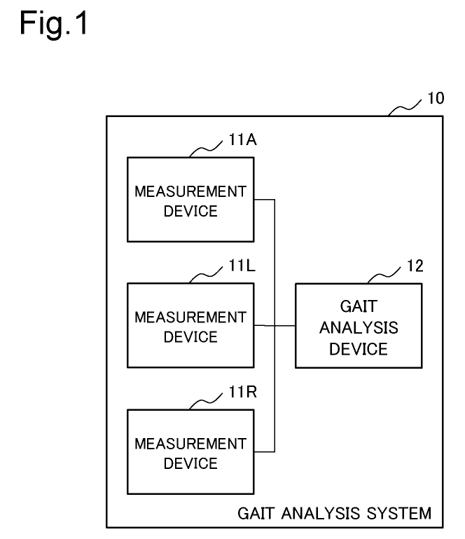

FIG. 1 is a block diagram illustrating a configuration of the gait analysis system in the present disclosure. A gait analysis system 10 includes a measurement device 11A (first measurement device), a measurement device 11L (second measurement device), a measurement device 11R (third measurement device), and a gait analysis device 12. The measurement device 11A, the measurement device 11L, and the measurement device 11R are devices having the same specifications. In a case where the measurement device 11A, the measurement device 11L, and the measurement device 11R are not distinguished from each other, they may each be referred to as a measurement device 11. A function of the gait analysis device 12 is installed on a mobile terminal used by the user. For example, the function of the gait analysis device 12 may be implemented in a server or a cloud accessible from the mobile terminal used by the user. Hereinafter, configurations of the measurement device 11 and the gait analysis device 12 will be individually described.

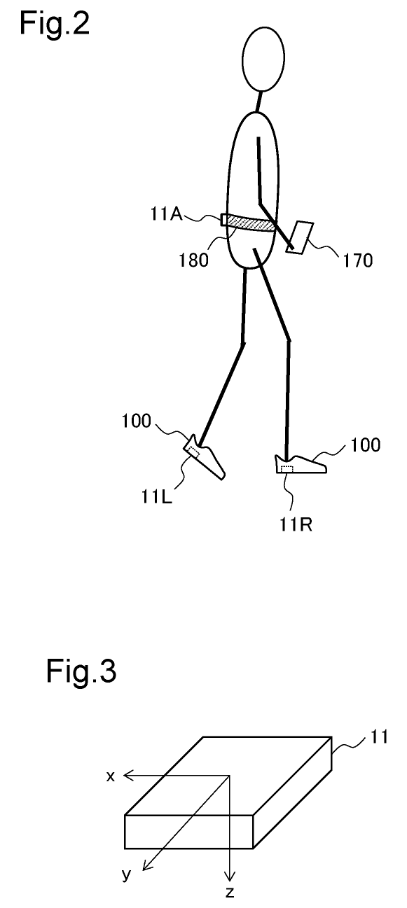

FIG. 2 is a conceptual diagram illustrating an arrangement example of the measurement device included in the gait analysis system in the present disclosure. FIG. 2 is a diagram of the user wearing the measurement device 11A, the measurement device 11L, and the measurement device 11R as viewed from a side viewpoint. The measurement device 11A is arranged on the waist of the user by use of a belt 180. The belt 180 may be a dedicated product for wearing the measurement device 11A or a general-purpose product. The measurement device 11A only needs to be arranged along the body axis of the user. For example, the measurement device 11A may be arranged at a part along the back, abdomen, spine, cervical spine, neck, or the like of the user. The measurement device 11L is arranged on footwear 100 of the left foot worn by the user. The measurement device 11R is arranged on the footwear 100 of the right foot worn by the user.

FIG. 3 is a conceptual diagram illustrating an example of a coordinate system set in the measurement device of the present disclosure. A common local coordinate system (x-axis, y-axis, z-axis) is set in the measurement device 11 (measurement device 11A, measurement device 11L, measurement device 11R). The measurement device 11 has a flat shape that is thin along the z-axis. The positive and negative directions of the x-axis, the y-axis, and the z-axis are arbitrarily set.

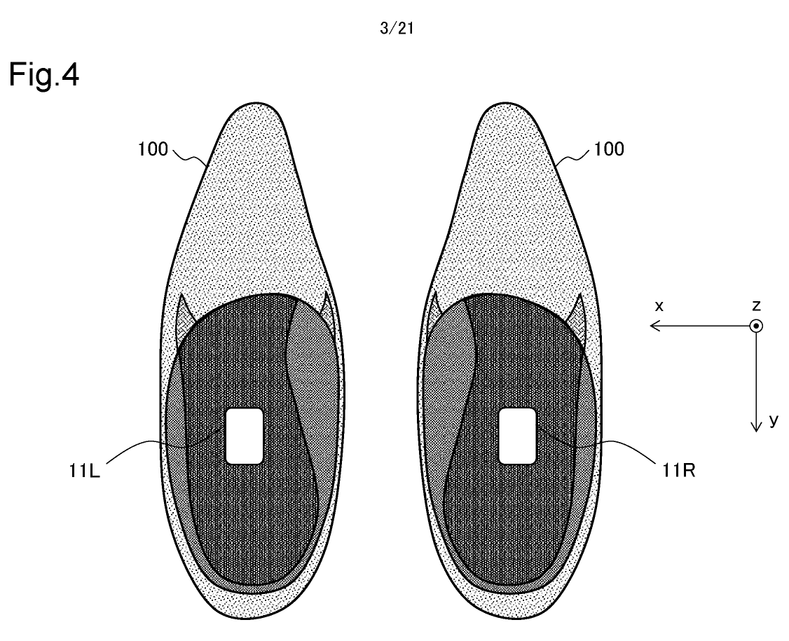

FIG. 4 is a conceptual diagram illustrating an arrangement example of the measurement device in the present disclosure. In the example of FIG. 4, the measurement device 11L and the measurement device 11R are each arranged at a position that comes in contact with the sole side of the foot. For example, the measurement device 11L and the measurement device 11R are each arranged below the arch of the foot. For example, the measurement device 11L and the measurement device 11R are each arranged on an insole inserted into the footwear 100. For example, the measurement device 11L and the measurement device 11R may each be arranged on the bottom surface of the footwear 100. For example, the measurement device 11L and the measurement device 11R may each be embedded in the body of the footwear 100. The measurement device 11L and the measurement device 11R may each be detachable from the footwear 100 or may each not be detachable from the footwear 100. The measurement device 11L and the measurement device 11R may each be installed at a position other than the back side of the foot arch as long as sensor data related to the movement of the foot can be measured. The measurement device 11L and the measurement device 11R may each be installed on a sock worn by the user or a decorative article such as an anklet worn by the user. The measurement device 11L and the measurement device 11R may each be directly pasted to the foot or may be embedded in the foot. Any one of the measurement device 11L and the measurement device 11R may be arranged in the footwear 100 on one side as long as data can be measured with which gait analysis can be performed. In a case where data of both feet is required, it is preferable that the measurement device 11L and the measurement device 11R are respectively arranged on the left footwear 100 and the right footwear 100.

The vertical directions (z-axis direction) of the measurement device 11L and the measurement device 11R are the same. In the x axis, the left side is positive. In the y axis, the front side is positive. In the z-axis, the upper side is positive. That is, the three axes of the local coordinate system set in the sensor data derived from the left foot and the three axes of the local coordinate system set in the sensor data derived from the right foot are the same.

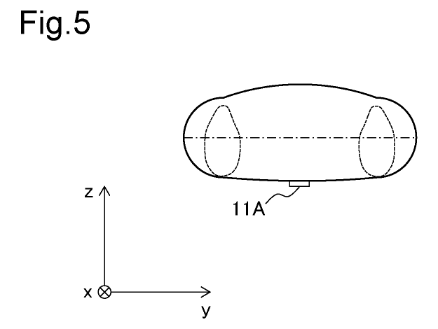

FIG. 5 is a conceptual diagram illustrating an arrangement example of the measurement device in the present disclosure. FIG. 5 illustrates a cross section of the waist of the user wearing the measurement device 11A. In FIG. 5, a position of a foot is indicated by a dotted line. In the example of FIG. 5, the measurement device 11A is arranged on the waist. The measurement device 11A is arranged at a part along the body axis of the user. For example, the measurement device 11A may be arranged at a part along the back, abdomen, spine, cervical spine, neck, or the like of the user. The measurement device 11A is arranged so that the z-axis is oriented in a traveling direction. In the x axis, the lower side is positive. In the y axis, the right side is positive. In the z-axis, the front side is positive.

### Measurement Device

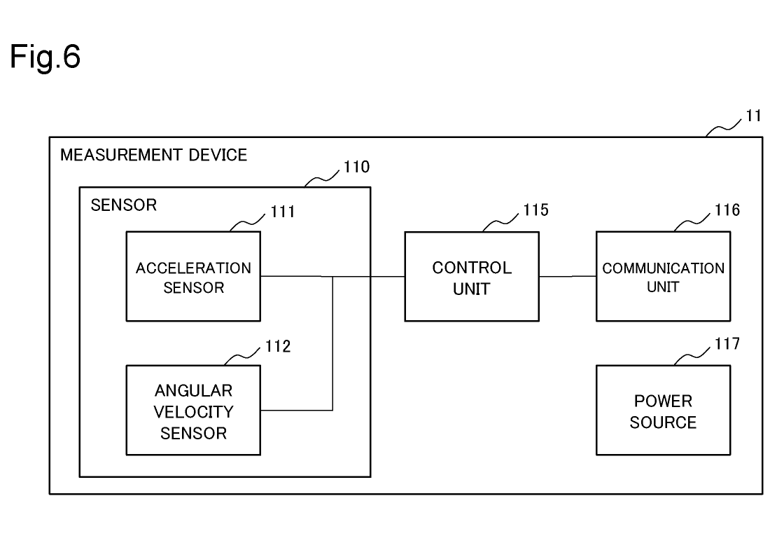

FIG. 6 is a block diagram illustrating an example of a configuration of the measurement device in the present disclosure. The measurement device 11 includes a sensor 110, a control unit 115, a communication unit 116, and a power source 117. The sensor 110 includes an acceleration sensor 111 and an angular velocity sensor 112. The sensor 110 may include a sensor other than the acceleration sensor 111 and the angular velocity sensor 112. Sensors other than the acceleration sensor 111 and the angular velocity sensor 112 that can be included in the sensor 110 will not be described.

The acceleration sensor 111 is a sensor that measures accelerations (also referred to as spatial accelerations) in three axial directions. The acceleration sensor 111 measures an acceleration as a physical quantity related to the movement of the foot. The acceleration sensor 111 outputs the measured acceleration to the control unit 115. For example, a sensor of a piezoelectric type, a piezoresistive type, a capacitance type, or the like can be used as the acceleration sensor 111. The sensor used as the acceleration sensor 111 is not limited as long as the sensor can measure an acceleration.

The angular velocity sensor 112 is a sensor that measures angular velocities (also referred to as spatial angular velocities) around three axes. The angular velocity sensor 112 measures an angular velocity as a physical quantity related to the movement of the foot. The angular velocity sensor 112 outputs the measured angular velocity to the control unit 115. For example, a sensor of a vibration type, a capacitance type, or the like can be used as the angular velocity sensor 112. The sensor used as the angular velocity sensor 112 is not limited as long as the sensor can measure an angular velocity.

The sensor 110 is implemented by, for example, an inertial measurement device that measures an acceleration and an angular velocity. An example of the inertial measurement device is an Inertial Measurement Unit (IMU). The IMU includes the acceleration sensor 111 that measures accelerations in three axial directions and the angular velocity sensor 112 that measures the angular velocities around three axes. The sensor 110 may be implemented by an inertial measurement device such as a Vertical Gyro (VG) or an Attitude Heading Reference System (AHRS). The sensor 110 may be implemented by a device other than the inertial measurement device as long as the sensor 110 can measure a physical quantity related to the movement of the foot.

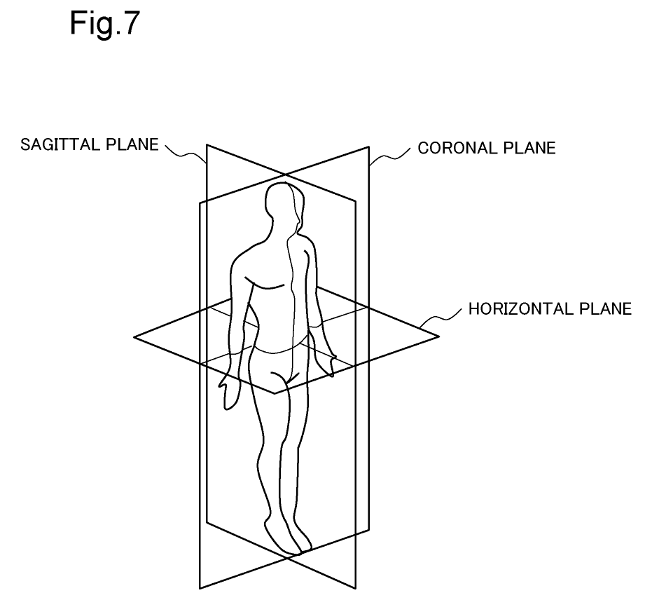

FIG. 7 is a conceptual diagram for describing a human body plane set for a human body. The human body plane includes a sagittal plane, a coronal plane, and a horizontal plane. The sagittal plane is a human body plane that divides the body into right and left parts. The coronal plane is a human body plane that divides the body into front and rear parts. The horizontal plane is a human body plane that horizontally divides the body.

The control unit 115 causes the acceleration sensor 111 and the angular velocity sensor 112 to measure sensor data. For example, the control unit 115 causes the acceleration sensor 111 and the angular velocity sensor 112 to start measurement in response to a measurement start signal transmitted from the gait analysis device 12. For example, the control unit 115 may cause the acceleration sensor 111 and the angular velocity sensor 112 to start measurement at a timing at which walking by the user is detected. For example, after the heights of both feet in the vertical direction are the same over a predetermined period set in advance, the control unit 115 starts the measurement of the sensor data from a time point at which movement of one of the right and left feet in the traveling direction is detected as a start point. The control unit 115 may be configured to start the measurement of the sensor data at a predetermined timing set in advance.

The control unit 115 acquires accelerations in three axial directions from the acceleration sensor 111. The control unit 115 acquires the angular velocities around three axes from the angular velocity sensor 112. For example, the control unit 115 performs Analog-to-Digital Conversion (AD conversion) on the acquired physical quantities (analog data) such as the angular velocity and acceleration. For example, the control unit 115 is provided with an AD conversion circuit that converts analog data into digital data. The physical quantities (analog data) measured by the acceleration sensor 111 and the angular velocity sensor 112 may be converted into digital data respectively in each the acceleration sensor 111 and the angular velocity sensor 112. The control unit 115 outputs the converted digital data (sensor data) to the communication unit 116. The control unit 115 synchronizes acceleration data and angular velocity data included in the sensor data with each other. The control unit 115 may temporarily store the sensor data in a storage unit (not illustrated).

The sensor data includes acceleration data and angular velocity data converted into digital data. The acceleration data includes acceleration vectors in three axial directions. The angular velocity data includes angular velocity vectors around three axes. The acceleration data and the angular velocity data are each associated with a measurement time of the data. Measurement times of the sensor data respectively measured by the measurement device 11A, the measurement device 11L, and the measurement device 11R are synchronized with each other by the gait analysis device 12. The control unit 115 may be configured to add corrections such as implementation error correction, temperature correction, and linearity correction to the acceleration data and the angular velocity data. For example, the control unit 115 is implemented by a microcomputer or a microcontroller that performs overall control and data processing of the measurement device 11. For example, the control unit 115 includes a Central Processing Unit (CPU), a Random Access Memory (RAM), a Read Only Memory (ROM), a flash memory, and the like.

The communication unit 116 acquires sensor data from the control unit 115. The communication unit 116 transmits the acquired sensor data to the gait analysis device 12. The sensor data transmitted from the communication unit 116 is received by the gait analysis device 12. A transmission timing of the sensor data is not particularly limited. For example, the communication unit 116 transmits sensor data at a transmission timing set in advance. For example, the communication unit 116 transmits the sensor data in real time in response to the measurement of the sensor data. For example, the communication unit 116 may store sensor data measured during a predetermined period and collectively transmit the stored sensor data at a timing set in advance. For example, the communication unit 116 may be configured to receive a measurement start signal from the gait analysis device 12. In this case, the communication unit 116 outputs the received measurement start signal to the control unit 115.

For example, the communication unit 116 transmits sensor data to the gait analysis device 12 via wireless communication. For example, the communication unit 116 transmits sensor data to the gait analysis device 12 via a wireless communication function (not illustrated) conforming to a standard such as Bluetooth (registered trademark) or WiFi (registered trademark). The communication function of the communication unit 116 may conform to a standard other than Bluetooth (registered trademark) or WiFi (registered trademark). The communication unit 116 may be configured to transmit sensor data to the gait analysis device 12 via a cable in a wired manner.

The power source 117 is a battery that supplies power for the measurement device 11 to operate. For example, the power source 117 is implemented by a thin battery such as a coin type or a button type. For example, the power source 117 is implemented by a primary battery such as a lithium primary battery, a silver oxide battery, an alkaline button battery, or an air zinc battery. In the case of being implemented by the primary battery, the power source 117 is preferably implemented by a long-life battery. The power source 117 may be implemented by a rechargeable secondary battery. In the case of being implemented by the secondary battery, the power source 117 may be a battery that can be charged in a wired manner or may be a battery to which wireless power supply can be performed. If the measurement device 11 is stacked on a wireless power supply device, the measurement device 11 can be charged appropriately when not in use.

### Gait Analysis Device

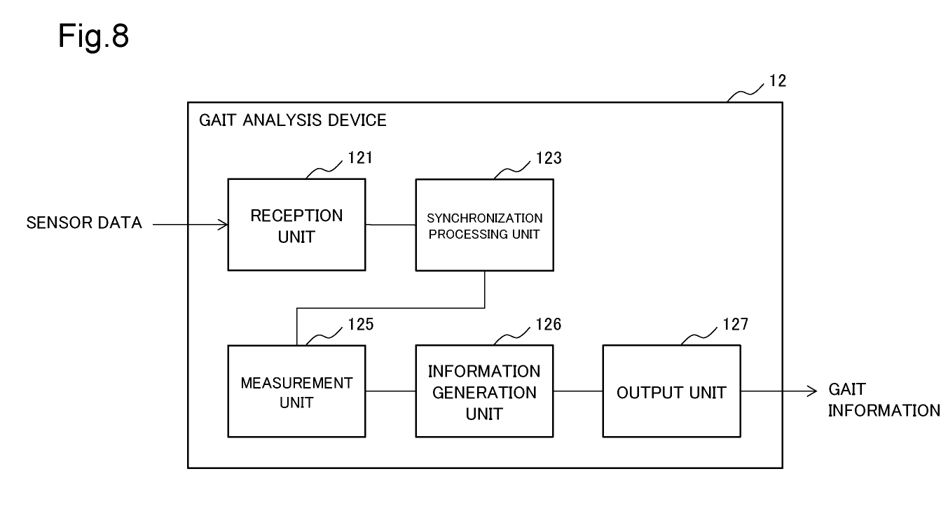

FIG. 8 is a block diagram illustrating an example of a configuration of a gait analysis device in the present disclosure. The gait analysis device 12 includes a reception unit 121, a synchronization processing unit 123, a measurement unit 125, an information generation unit 126, and an output unit 127.

The reception unit 121 receives pieces of sensor data from the measurement device 11A, the measurement device 11L, and the measurement device 11R. The sensor data includes acceleration data in three axial directions and angular velocity data around three axes. The measurement time is associated with the sensor data. The reception unit 121 acquires the sensor data via wireless or wired communication. The reception unit 121 transmits the received sensor data to the synchronization processing unit 123. For example, the acceleration data in three axial directions and the angular velocity data around three axes included in the sensor data acquired from each of the measurement device 11A, the measurement device 11L, and the measurement device 11R are input to a buffer memory (not illustrated) of a processor. The buffer memory (not illustrated) is individually allocated to each of the measurement device 11A, the measurement device 11L, and the measurement device 11R. For each buffer memory, peak detection is performed with a window size equal to or less than 10 (for example, 7).

The synchronization processing unit 123 synchronizes the pieces of the sensor data transmitted from the reception unit 121 with each other on the time axis. The synchronization processing unit 123 corrects the time deviation, based on the measurement time of the sensor data, and arranges the sensor data on a common time axis. The synchronization processing by the synchronization processing unit 123 temporally aligns the pieces of the sensor data obtained from the measurement device 11A, the measurement device 11L, and the measurement device 11R arranged at different positions. The synchronization processing unit 123 provides the synchronized sensor data to the measurement unit 125.

The measurement unit 125 measures a gait parameter by using the synchronized sensor data provided from the synchronization processing unit 123. The gait parameter is an index quantitatively indicating a walking motion. For example, the measurement unit 125 calculates the gait parameter by using normalized time-series data (gait waveform) of the sensor data.

The measurement unit 125 measures a double support time by using the sensor data measured by the measurement device 11A arranged on the waist. The measurement unit 125 specifies the left and right feet by using traveling direction accelerations and the angular velocities around the traveling direction axis measured by the measurement device 11L arranged on the footwear of the left foot and the measurement device 11R arranged on the footwear of the right foot. The measurement unit 125 measures the double support time corresponding to a time during which both feet are simultaneously in contact with the ground with respect to the specified left and right feet. In addition, the measurement unit 125 measures a load time corresponding to a time from when the heel touches the ground to when the toe touches the ground in a stance phase with respect to the specified left and right feet. The measurement unit 125 may be configured to calculate a time required for one step, a cadence indicating the number of steps per minute, a walking speed, a left-right difference or balance of walking, and the like.

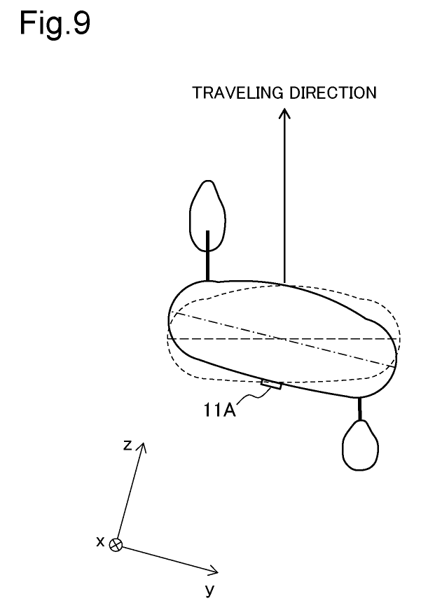

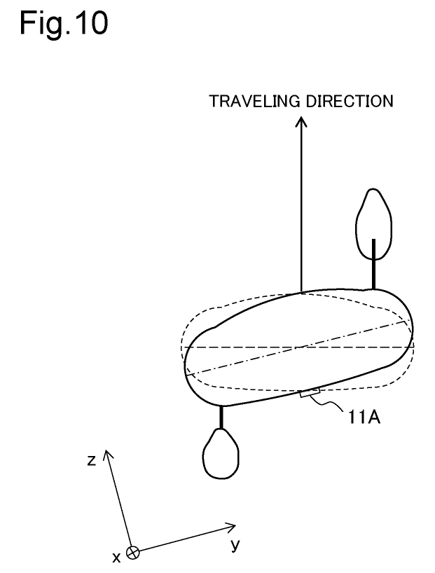

FIGS. 9-10 are conceptual diagrams for describing a change in the direction of the measurement device in the present disclosure. FIGS. 9-10 each illustrate a cross section of the waist of the user wearing the measurement device 11A. The user is walking in the traveling direction. In the example of FIG. 9, the left foot is in front. The z-axis of the measurement device 11A is inclined to the right side with respect to the traveling direction, in the horizontal plane. In the example of FIG. 10, the right foot is in front. The z-axis of the measurement device 11A is inclined to the left side with respect to the traveling direction, in the horizontal plane. The direction of the measurement device 11A changes periodically according to walking of the user.

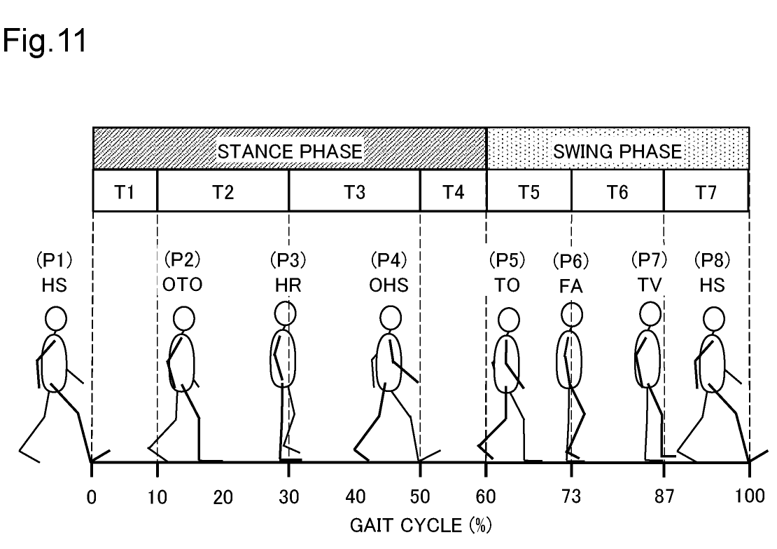

FIG. 11 is a conceptual diagram for describing one gait cycle with the right foot as a reference. One gait cycle with the left foot as a reference is also similar to that of the right foot. The horizontal axis in FIG. 11 indicates one gait cycle of the right foot with a time point at which the heel of the right foot lands on the ground as a start point and a time point at which the heel of the right foot next lands on the ground as an end point. The horizontal axis in FIG. 11 is normalized with one gait cycle as 100%. Performing normalization with one gait cycle as 100% is referred to as first normalization. The one gait cycle of one foot is roughly divided into a stance phase in which at least a part of the sole side of the foot is in contact with the ground and a swing phase in which the sole side of the foot is separated from the ground. The stance phase is a period in which at least a part of the sole side of the foot is in contact with the ground. The stance phase is further subdivided into an initial stance period T1, a mid-stance period T2, a terminal stance period T3, and a pre-swing period T4. The swing phase is a period in which the sole side of the foot is separated from the ground. The swing phase is further subdivided into an initial swing period T5, a mid-swing period T6, and a terminal swing period T7. The horizontal axis in FIG. 11 is normalized so that the stance phase is 60% and the swing phase is 40%. Performing normalization of the gait waveform so that the stance phase is 60% and the swing phase is 40% is referred to as second normalization. The periods illustrated in FIG. 11 are examples, and the periods constituting one gait cycle, the names of those periods, and the like are not limited.

As illustrated in FIG. 11, a plurality of events occur in walking. In the walking, a plurality of events in the walking are also referred to as gait events. P1 represents an event (heel strike) in which the heel of the right foot touches the ground (HS: Heel Strike). P2 represents an event (opposite toe off) in which the toe of the left foot is separated from the ground in a state where the sole of the right foot is grounded (OTO: Opposite Toe Off). P3 represents an event (heel rise) in which the heel of the right foot rises in a state where the sole of the right foot is grounded (HR: Heel Rise). P4 is an event (opposite heel strike) in which the heel of the left foot is grounded (OHS: Opposite Heel Strike). P5 represents an event (toe off) in which the toe of the right foot is separated from the ground in a state where the sole of the left foot is grounded (TO: Toe Off). P6 represents an event (foot adjacent) in which the left foot and the right foot cross each other in a state where the sole of the left foot is grounded (FA: Foot Adjacent). P7 represents an event (tibia vertical) in which the tibia of the right foot is approximately perpendicular to the ground in a state where the sole of the left foot is grounded (TV: Tibia Vertical). P8 represents an event (heel strike) in which the heel of the right foot touches the ground (HS: Heel Strike). P8 is relevant to the end point of the gait cycle starting from P1 and is relevant to the start point of the next gait cycle. The gait events illustrated in FIG. 11 are examples, and do not limit the events that occur in the walking or the names of those events. For example, the events that occur in the walking include a gait event such as a Toe Strike (TS).

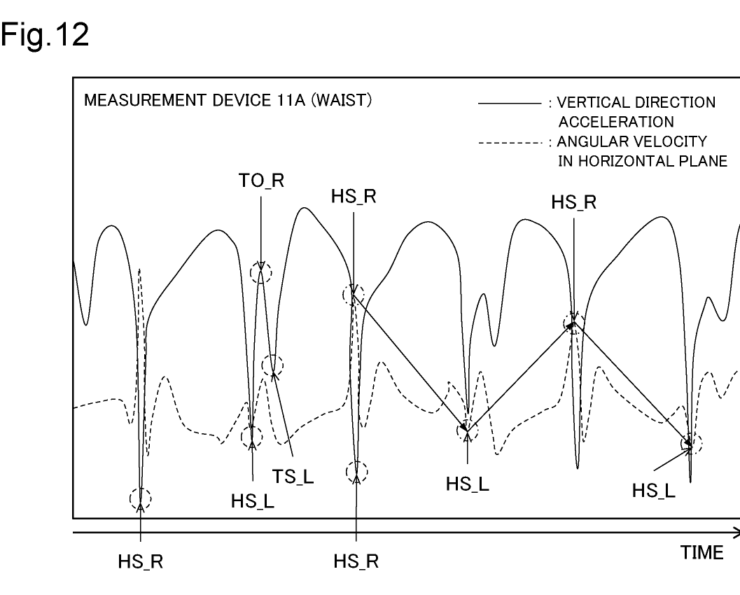

FIGS. 12-15 are graphs illustrating examples of sensor data measured by the measurement device of the present disclosure. Pieces of sensor data measured by the measurement device 11A, the measurement device 11L, and the measurement device 11R are measured according to walking of the user. In the time-series data of the sensor data, features regarding the gait of the user periodically appear.

FIG. 12 illustrates an example of time-series data of each of a vertical direction acceleration and an angular velocity in the horizontal plane measured by the measurement device 11A worn on the waist of the user. The vertical direction acceleration is an acceleration in the x-axis direction (vertical direction) set in the measurement device 11A. The angular velocity in the horizontal plane is an angular velocity around the x-axis set in the measurement device 11A. In the time-series data (solid line) of the vertical direction acceleration and the time-series data (broken line) of the angular velocity in the horizontal plane, a plurality of peaks appear. In FIG. 12, minimum points and maximum points of the peaks relevant to the gait events such as a heel strike HS_R of the right foot, a heel strike HS_L of the left foot, a toe off TO_R of the right foot, and a toe strike TS_L of the left foot are clearly indicated by being surrounded by broken line circles.

In FIG. 12, in the time-series data (solid line) of the vertical direction acceleration, a minimum value relevant to a timing of the heel strike HS_L of the left foot and a minimum value relevant to a timing of the heel strike HS_R of the right foot alternately appear. In the time-series data (solid line) of the vertical direction acceleration, timings of the heel strikes HS can be determined by those minimum values, but it cannot be determined whether they are of the left foot or the right foot. On the other hand, in the time-series data (broken line) of the angular velocity in the horizontal plane, a minimum value relevant to a timing of the heel strike HS_L of the left foot and a maximum value relevant to a timing of the heel strike HS_R of the right foot alternately appear. Although not illustrated, the minimum value relevant to the timing of the heel strike HS_L of the left foot is negative, and the maximum value relevant to the timing of the heel strike HS_R of the right foot is positive. For that reason, the heel strike HS_L of the left foot and the heel strike HS_R of the right foot can be distinguished from each other depending on whether a peak value of the time-series data (broken line) of the angular velocity in the horizontal plane is positive or negative at a timing of the heel strike HS detected in the time-series data (solid line) of the vertical direction acceleration.

If the time-series data (solid line) of the vertical direction acceleration is used, the double support time (DST) can be measured. The double support time is a time during which both feet are simultaneously in contact with the ground. The double support time is individually defined in a state where the left foot or the right foot is stepped forward. The double support time is related to exercise ability such as cadence, walking speed, and balance ability. For that reason, if the double support time can be measured, it is possible to estimate the exercise ability of the user.

In FIG. 12, a time difference from a time relevant to the heel strike HS_L of the left foot to a time relevant to the toe off TO_R of the right foot corresponds to the double support time in a state where the left foot is stepped forward. In FIG. 12, a time difference from a time relevant to the heel strike HS_R of the right foot to a time relevant to a toe off TO_L of the left foot corresponds to the double support time in a state where the right foot is stepped forward.

If the time-series data (solid line) of the vertical direction acceleration is used, the load time can be measured. The load time is a time from when the heel touches the ground to when the toe touches the ground in the stance phase. The load time is defined for each of the left foot and the right foot. The load time is related to ability to support the body with the heel during walking. For that reason, if the load time can be measured, it is possible to estimate the ability of the user to support the body with the heel during walking.

In FIG. 12, a time difference from the time relevant to the heel strike HS_L of the left foot to a time relevant to the toe strike TS_L of the left foot corresponds to the load time of the left foot. Although not illustrated in FIG. 12, a time difference from the time relevant to the heel strike HS_R of the right foot to a time relevant to a toe strike TS_R of the right foot corresponds to the load time of the right foot.

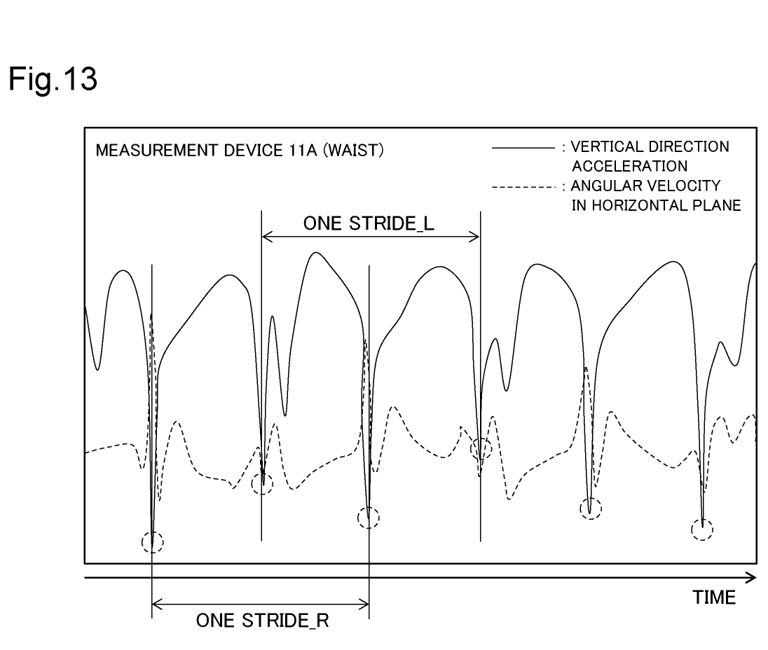

FIG. 13 illustrates an example of time-series data of each of the vertical direction acceleration and the angular velocity in the horizontal plane measured by the measurement device 11A worn on the waist of the user. FIG. 13 is an example in which different information is illustrated in the same graph as FIG. 12. In FIG. 13, minimum values of peaks relevant to the heel strike HS_L of the left foot and the heel strike HS_R of the right foot are clearly indicated by being surrounded by broken line circles. A time difference between temporally continuous heel strikes HS_L of the left foot corresponds to the time for one stride_L of the left foot. A time difference between temporally continuous heel strikes HS_R of the right foot corresponds to the time for one stride_R of the right foot.

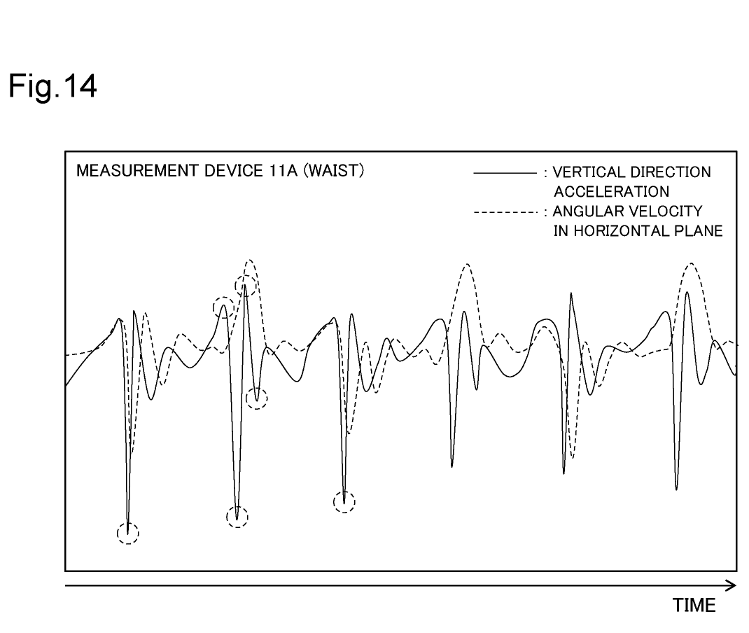

FIG. 14 illustrates an example of time-series data of each of a traveling direction acceleration and an angular velocity in the coronal plane measured by the measurement device 11A worn on the waist of the user. The traveling direction acceleration is an acceleration in the z-axis direction (front-rear direction) set in the measurement device 11A. The angular velocity in the coronal plane is an angular velocity around the z-axis set in the measurement device 11A. In the time-series data (solid line) of the traveling direction acceleration and the time-series data (broken line) of the angular velocity in the coronal plane, a plurality of peaks associated with gait events appear.

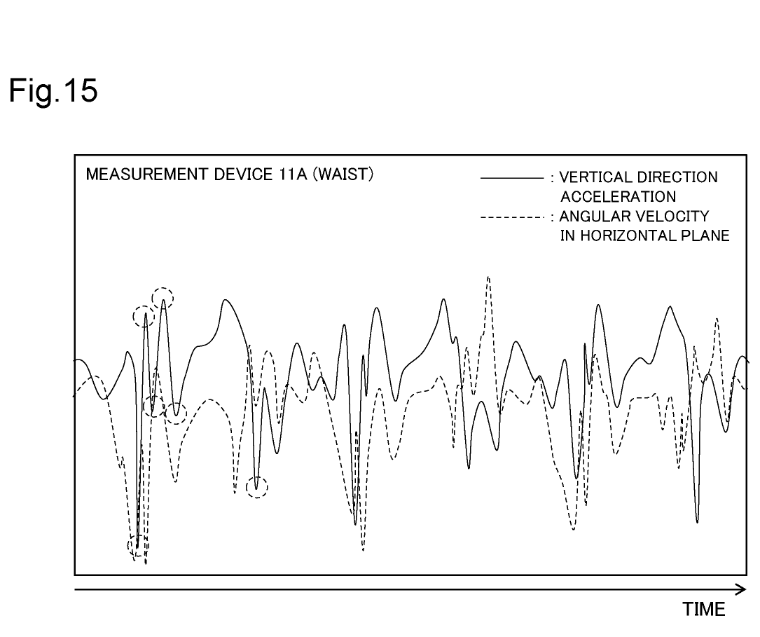

FIG. 15 illustrates an example of time-series data of each of a lateral direction acceleration and an angular velocity in the sagittal plane measured by the measurement device 11A worn on the waist of the user. The lateral direction acceleration is an acceleration in the y-axis direction (left-right direction) set in the measurement device 11A. The angular velocity in the sagittal plane is an angular velocity around the y-axis set in the measurement device 11A. In the time-series data of the lateral direction acceleration (solid line) and the time-series data of the angular velocity in the sagittal plane (broken line), a plurality of peaks associated with gait events appear. For example, if extreme values of peaks similar to those in the example of FIG. 12 are detected, the double support time and the load time can be measured.

In a case where pieces of the sensor data measured by the measurement device 11L and the measurement device 11R are used, a feature used for measurement of the double support time appears at an inflection point of time-series data regarding a trajectory of the foot calculated by use of the pieces of the sensor data. In a case where a change in the trajectory is gentle, an inflection point in the trajectory during one stride cannot be detected, and an excessive value is output or the measurement time reaches a limit, so that an abnormal value is likely to be output. For example, in the case of a person with weak gait, the gait variation is gentle, and thus, a frequency of outputting the abnormal value is high. In the present example embodiment, since the sensor data measured by the measurement device 11A arranged on the waist is used, the double support time can be accurately measured even for the person with weak gait.

The measurement unit 125 measures various gait parameters by using pieces of the time-series data of the spatial acceleration and the spatial angular velocity measured by the measurement device 11A, the measurement device 11L, and the measurement device 11R. The measurement unit 125 may be configured to measure the gait parameters by using integral values or second-order integral values of the spatial acceleration and the spatial angular velocity.

The measurement unit 125 measures various gait parameters by using pieces of the sensor data measured by the measurement device 11L and the measurement device 11R respectively arranged on the left footwear and the right footwear. The pieces of the sensor data measured by the measurement device 11L and the measurement device 11R respectively arranged on the left footwear and the right footwear are temporally synchronized with each other by synchronization processing. For example, the measurement unit 125 calculates posture angles of the measurement device 11L and the measurement device 11R by using a Madgwick filter. For example, the measurement unit 125 calculates the gait parameters related to a distance, a height, an angle, a speed, a time, a Center of Pressure Exclusion Index (CPEI), a frailty level, and the like. The gait parameters are used for generation of information regarding a physical condition, physical ability, and the like. The gait parameters calculated by the measurement unit 125 are not particularly limited.

The gait parameter related to the distance and the height includes a stride length, an outward turning distance, a foot raising height, Foot Clearance (FTC), and Minimum Toe Clearance (MTC). The stride length indicates a distance between a front foot and a rear foot during walking. The outward turning distance indicates a maximum value of a distance at which the foot is separated outward with respect to the traveling direction in the swing phase. The foot raising height indicates a maximum value of a distance between the measurement device 11L or the measurement device 11R and the ground in the swing phase. The FTC indicates a maximum value of a distance between the heel and the ground in the swing phase. The MTC indicates a minimum value of a distance between the toe and the ground in the swing phase.

The gait parameter related to the angle includes a ground contact angle, a takeoff angle, a toe direction, a roll angle of a heel strike, a roll angle of a toe off, a swing peak angular velocity, and a hallux angle. The ground contact angle indicates a maximum value of an angle formed by the sole surface and the ground at the time of the heel strike. The takeoff angle indicates an angle formed by the sole surface and the ground in the swing phase. The toe direction indicates an average value of the toe directions with respect to the traveling direction in the swing phase. The roll angle of the heel strike is an angle formed by the ankle and the ground at the time of the heel strike as viewed from the rear viewpoint. The roll angle of the toe off is an angle formed by the ankle and the ground at the time of kicking as viewed from the rear viewpoint. The swing peak angular velocity is an angular velocity in the ankle joint dorsiflexion direction in a section from immediately after kicking until the toe comes into closest contact with the ground. The hallux angle indicates an angle at which the big toe of the foot is inclined toward the second toe. Specifically, the hallux angle is an angle formed by the center line of the first metatarsal and the center line of the first proximal phalanx.

The gait parameter related to the speed includes a walking speed, cadence, and a maximum speed in swing. The walking speed indicates a speed in the walking. The cadence indicates the number of steps per minute. The maximum speed in swing indicates a speed at which the foot is swung in the swing phase.

The gait parameter related to the time includes a stance time, a load time, a plantar ground contact time, a kicking time, a swing time, and a double support time (DST). The stance time indicates a time during which the foot is in contact with the ground during walking. The stance time is a sum of the load time, the plantar ground contact time, and the kicking time. The load time is a time from when the heel touches the ground to when the toe touches the ground in the stance phase. The plantar ground contact time is a time during which the entire plantar surface is grounded and the plantar surface and the ground are horizontal in the stance phase. The kicking time is a time until the toe kicks the ground from the state of the plantar ground contact in the stance phase. The swing time indicates a time during which the foot is separated from the ground during walking. The DST is divided into DST1 and DST2. The DST1 indicates a time during which the foot on which the measurement device 11L or the measurement device 11R is implemented is in front of the opposite foot in a period in which both feet are simultaneously in contact with the ground. The DST2 indicates a time during which the foot on which the measurement device 11L or the measurement device 11R is implemented is behind the opposite foot in a period in which both feet are simultaneously in contact with the ground.

The CPEI indicates an estimated value of the expansion ratio of the movement of the center of foot pressure applied to the ground during a period of the stance phase. The frailty level is an estimated value of frailty detected from the walking state. For example, the measurement unit 125 estimates a possibility of frailty as the frailty level. In a case where there is no possibility of frailty, the measurement unit 125 outputs a determination result indicating that there is no frailty. In a case where there is a possibility of frailty, the measurement unit 125 outputs a determination result indicating that there is a possibility of frailty. In a case where the possibility of frailty is high, the measurement unit 125 outputs a determination result indicating that the possibility of frailty is high.

The information generation unit 126 generates gait information according to walking of the user by using the measurement value and gait parameter measured by the measurement unit 125. For example, the information generation unit 126 generates the gait information including the measurement value and the gait parameter. For example, the information generation unit 126 generates the gait information including an evaluation result according to the measurement value and the gait parameter. For example, the information generation unit 126 generates the gait information by using a machine learning model trained to output an evaluation result regarding the body such as the physical condition and the physical ability according to inputs of the measurement value and the gait parameter. For example, the information generation unit 126 generates the gait information according to the measurement value and the gait parameter by using a generative AI. For example, the information generation unit 126 generates the gait information including advice according to the evaluation result of the physical condition or the physical ability. The gait information generated by the information generation unit 126 is not particularly limited as long as it is information that can be generated by use of the measurement value and the gait parameter measured by the measurement unit 125.

For example, the information generation unit 126 evaluates non-uniformity such as a left-right difference or balance of the gait ability of the user by using the sensor data measured by the measurement device 11A arranged on the waist. Specifically, the information generation unit 126 evaluates the left-right difference or balance of the gait ability according to a difference between positive and negative maximum values, with respect to the angular velocity or angle around the x-axis of the measurement device 11A. For example, in a case where the user is looked down from above and the counterclockwise angular velocity or angle is larger than the clockwise angular velocity or angle, it can be determined that the movement to the left side is large with the body axis as the center. For example, in a case where the user is looked down from above and the clockwise angular velocity or angle is larger than the counterclockwise angular velocity or angle, it can be determined that the movement to the right side is large with the body axis as the center. For example, the information generation unit 126 is configured to determine that the left-right difference or balance of the gait ability is misaligned in a case where the angular velocity or angle exceeds a determination threshold set in advance, with respect to the angular velocity or angle around the body axis.

For example, the information generation unit 126 compares the measurement value and the gait parameter with a reference value and a normal value, and evaluates the physical condition and the physical ability such as walking stability, left-right balance, and walking speed of the user. The information generation unit 126 generates the gait information including an evaluation result that the measurement value and the gait parameter are normal for the user whose measurement value and gait parameter are better than the reference value and the normal value. The information generation unit 126 generates the gait information including an evaluation result that there is an abnormality for the user whose measurement value or gait parameter is inferior to the reference value or the normal value.

For example, the information generation unit 126 compares the current measurement value and gait parameter with the past data for the user to be evaluated, and evaluates the walking stability, the left-right balance, the walking speed, and the like. The information generation unit 126 generates the gait information including an evaluation result that the health condition is improved for the user whose measurement value or gait parameter is improved as compared with the past data. The information generation unit 126 generates the gait information including an evaluation result that the health condition is maintained for the user having no change in the measurement value or the gait parameter as compared with the past data. The information generation unit 126 generates the gait information including an evaluation result that the health condition has deteriorated for the user whose measurement value or gait parameter has deteriorated as compared with the past data.

For example, the information generation unit 126 outputs action recommendation information optimized in accordance with an evaluation result regarding the body such as the physical condition and physical ability of the user. The action recommendation information includes information for prompting the user to make a decision. For example, the output action recommendation information is displayed on a screen of a mobile terminal carried by the user. The user who has confirmed the action recommendation information displayed on the screen of the mobile terminal can take an action leading to improvement of the physical condition and physical ability by taking an action according to the information.

The output unit 127 outputs the information generated by the information generation unit 126. For example, the output unit 127 outputs the gait information to the mobile terminal used by the user. For example, the output unit 127 outputs the gait information to a terminal device or a server via the mobile terminal used by the user. For example, the output unit 127 may be configured to output the gait information to an external system or the like. For example, the gait information is displayed on the screen of the mobile terminal or the terminal device. The output unit 127 may be configured to perform display control of the screen of the mobile terminal or the terminal device. For example, the gait information is provided in a format that is easily grasped visually, such as numerical data, a graph, or text information.

For example, the gait analysis device 12 is implemented in a cloud or a server connected to the mobile terminal used by the user. The mobile terminal is a portable communication device. For example, the mobile terminal is a portable type communication device having a communication function, such as a smartphone, a smart watch, or a mobile phone. For example, the gait analysis device 12 is connected to the mobile terminal via wireless communication. For example, the gait analysis device 12 is connected to the mobile terminal via a wireless communication device (not illustrated) conforming to a standard such as Bluetooth (registered trademark) or WiFi (registered trademark). The wireless communication device may conform to a standard other than Bluetooth (registered trademark) or WiFi (registered trademark). The measurement value and gait parameter included in the gait information may be used by an application installed on the mobile terminal. For example, the mobile terminal executes processing using the measurement value and gait parameter by the application installed on the mobile terminal.

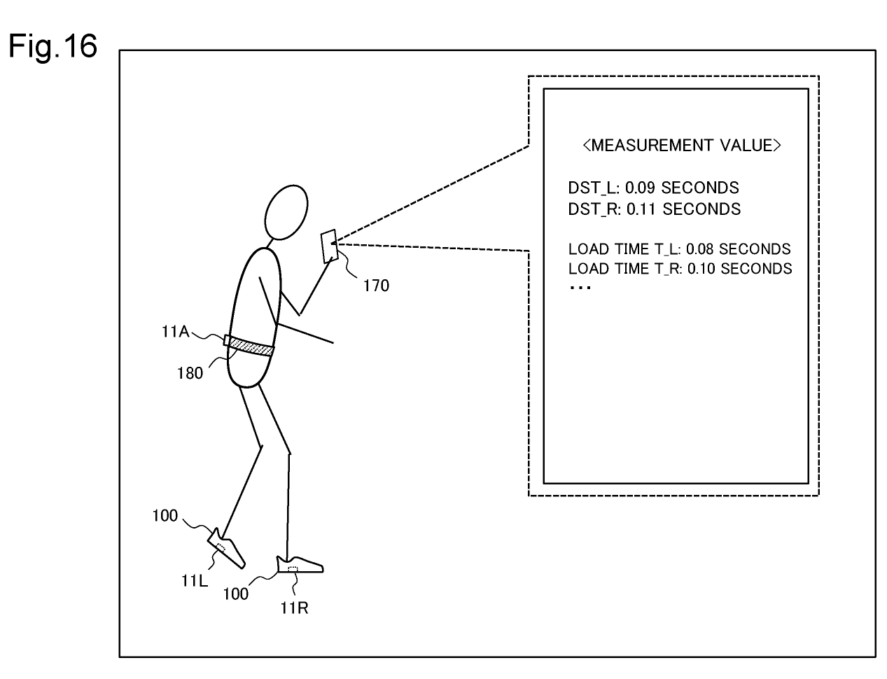

FIGS. 16-18 are conceptual diagrams illustrating display examples of gait information output from the gait analysis device in the present disclosure. The measurement device 11A is worn on the waist of the user by the belt 180. The user is walking while wearing a pair of the footwear 100 on which the measurement device 11L and the measurement device 11R are mounted. The measurement device 11A, the measurement device 11L, and the measurement device 11R are connected to a mobile terminal 170 by wireless communication. The measurement device 11A, the measurement device 11L, and the measurement device 11R are connected to the gait analysis device 12 implemented on a cloud or a server via the mobile terminal 170. The gait information output from the gait analysis device 12 is displayed on a screen of the mobile terminal 170 used by the user. The user can recognize the physical condition and physical ability of the user by confirming the gait information displayed on the screen of the mobile terminal 170.

FIG. 16 illustrates an example in which a measurement value measured in accordance with walking of the user is displayed on the screen of the mobile terminal. The screen of the mobile terminal 170 displays a double support time DST_L in a state where the left foot is stepped forward and a double support time DST_R in a state where the right foot is stepped forward. In addition, the screen of the mobile terminal 170 displays a load time T_L of the left foot and a load time T_R of the right foot. The user can recognize numerical values of the double support time and the load time by confirming the measurement values displayed on the screen of the mobile terminal 170.

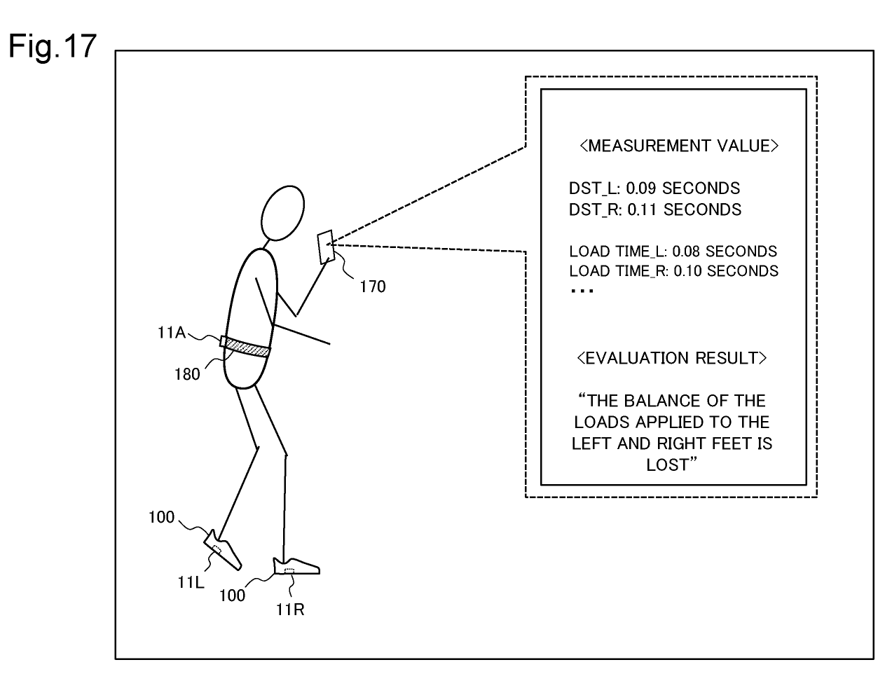

FIG. 17 illustrates an example in which a measurement value measured in accordance with walking of the user is displayed on the screen of the mobile terminal. The screen of the mobile terminal 170 displays a double support time DST_L in a state where the left foot is stepped forward and a double support time DST_R in a state where the right foot is stepped forward. In addition, the screen of the mobile terminal 170 displays a load time T_L of the left foot and a load time T_R of the right foot. Further, the screen of the mobile terminal 170 displays an evaluation result of “the balance of the loads applied to the left and right feet is lost”. The user can recognize a problem in the walking of the user by confirming the measurement value and evaluation result displayed on the screen of the mobile terminal 170.

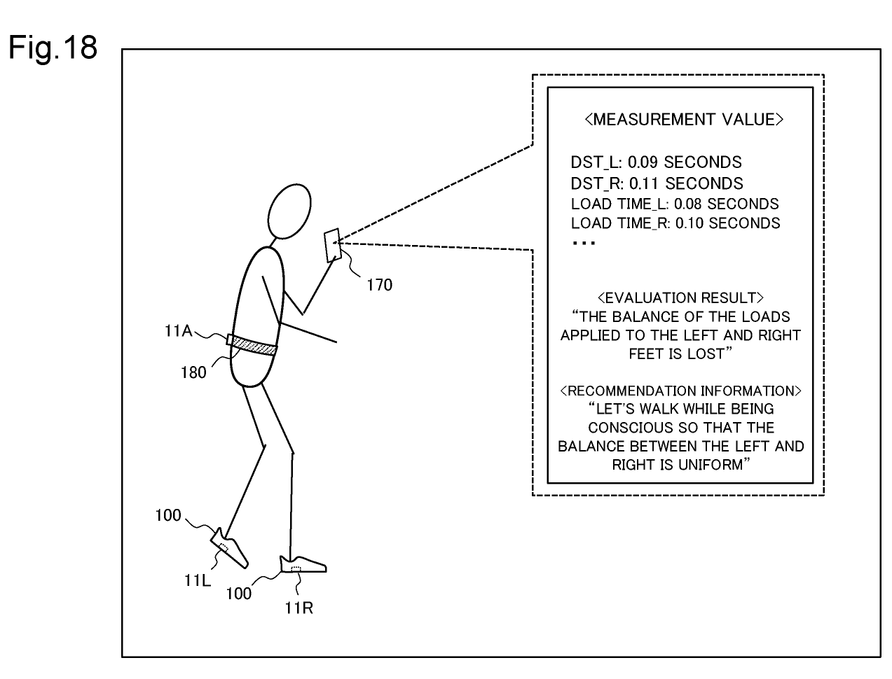

FIG. 18 illustrates an example in which a measurement value measured in accordance with walking of the user is displayed on the screen of the mobile terminal. The screen of the mobile terminal 170 displays a double support time DST_L in a state where the left foot is stepped forward and a double support time DST_R in a state where the right foot is stepped forward. In addition, the screen of the mobile terminal 170 displays a load time T_L of the left foot and a load time T_R of the right foot. In addition, the screen of the mobile terminal 170 displays an evaluation result of “the balance of the loads applied to the left and right feet is lost”. Further, the screen of the mobile terminal 170 displays advice according to the evaluation result regarding walking of the user, such as “Let's walk while being conscious so that the balance between the left and right is uniform”. The user can recognize that it is better to improve walking by confirming the advice displayed on the screen of the mobile terminal 170.

### Operation

Next, an example of an operation of the gait analysis device in the present disclosure will be described with reference to the drawings. The following description of the operation (gait analysis method) is schematic. Details of the following operation (gait analysis method) are as described in the above-described configuration.

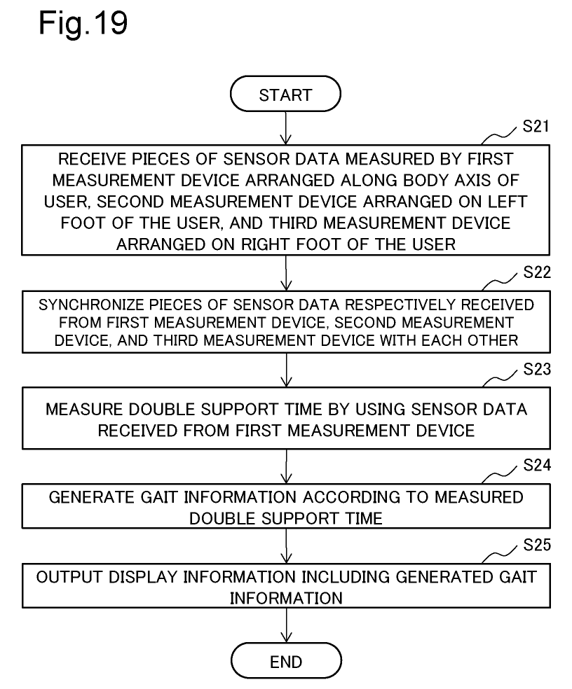

FIG. 19 is a flowchart illustrating an example of the operation of the gait analysis device in the present disclosure. In the description of the processing as per the flowchart in FIG. 19, a component of the gait analysis device 12 is assumed as an operating subject. The operating subject of the processing as per the flowchart in FIG. 19 may be the gait analysis device 12. For example, the processing as per the flowchart in FIG. 19 is implemented by a processor executing a program stored in a memory mounted in a computer (not illustrated) in which the function of the gait analysis device 12 is implemented.

In FIG. 19, the reception unit 121 receives pieces of sensor data measured by the first measurement device arranged along the body axis of the user, the second measurement device arranged on the left foot of the user, and the third measurement device arranged on the right foot of the user (step S11).

Next, the synchronization processing unit 123 synchronizes the pieces of the sensor data respectively received from the first measurement device, the second measurement device, and the third measurement device with each other (step S12).

Next, the measurement unit 125 measures a double support time by using the sensor data received from the first measurement device (step S13).

Next, the information generation unit 126 generates gait information according to the measured double support time (step S14).

Next, the output unit 127 outputs display information including the generated gait information (step S15).

As described above, the gait analysis system of the present example embodiment includes the first measurement device, the second measurement device, the third measurement device, and the gait analysis device. The first measurement device is arranged along the body axis of the user. The second measurement device is arranged on the left foot of the user. The third measurement device is arranged on the right foot of the user. The gait analysis device includes a reception unit, a synchronization processing unit, a measurement unit, an information generation unit, and an output unit. The reception unit receives pieces of sensor data measured by the first measurement device, the second measurement device, and the third measurement device. The synchronization processing unit synchronizes the pieces of the sensor data respectively received from the first measurement device, the second measurement device, and the third measurement device with each other. The measurement unit measures a double support time by using the sensor data received from the first measurement device. The information generation unit generates gait information according to the measured double support time. The output unit outputs display information including the generated gait information.

The gait analysis device of the present example embodiment synchronizes pieces of sensor data measured by the first measurement device arranged along the body axis of the user, the second measurement device arranged on the left foot of the user, and the third measurement device arranged on the right foot of the user with each other. In the case of using sensor data measured according to the movement of the left and right feet, it is necessary to detect an inflection point of time-series data of the sensor data. For the person with weak gait, since the curvature of the time-series data of the sensor data decreases and the sampling rate is low, the inflection point of the time-series data of the sensor data cannot be accurately detected. In the present example embodiment, the double support time is measured by use of the sensor data measured by the first measurement device arranged along the body axis of the user without use of the sensor data measured according to the movement of the left and right feet. For that reason, according to the present example embodiment, the double support time can be accurately measured by use of the sensor data measured by the first measurement device arranged along the body axis of the user.

In an aspect of the present example embodiment, the measurement unit detects a peak of the vertical direction acceleration received from the first measurement device. The measurement unit specifies the left and right feet relevant to the peak of the vertical direction acceleration depending on whether the angular velocity in the horizontal plane at the time of the extreme value of the detected peak of the vertical direction acceleration is positive or negative. The measurement unit measures, for each of the specified left foot and right foot, a time difference between a time of heel strike of one foot and a time of toe off of the other foot in the same gait cycle, as the double support time. According to the present aspect, by specifying the left and right feet by using the peak of the vertical direction acceleration received from the first measurement device and whether the angular velocity in the horizontal plane is positive or negative, it is possible to measure the double support time with high accuracy by using the sensor data measured by the single first measurement device.

In an aspect of the present example embodiment, the measurement unit measures, as the double support time in a state where the left foot is stepped forward, a time difference from a time relevant to the heel strike of the left foot to a time relevant to the toe off of the right foot by using the time-series data of the vertical direction acceleration received from the first measurement device. The measurement unit measures, as the double support time in a state where the right foot is stepped forward, a time difference from a time relevant to the heel strike of the right foot to a time relevant to the toe off of the left foot by using the time-series data of the vertical direction acceleration received from the first measurement device. According to the present aspect, by individually measuring the double support time in a state where each of the left foot and the right foot is stepped forward, it is possible to analyze the left-right difference in walking in more detail.

In an aspect of the present example embodiment, the measurement unit measures, as the load time, a time difference from a time relevant to heel strike to a time relevant to the toe strike for each of the left and right feet by using the time-series data of the vertical direction acceleration received from the first measurement device. According to the present aspect, by measuring the load time by using the vertical direction acceleration data measured by the first measurement device, it is possible to evaluate the stability of walking without using a measurement device of a foot portion.

In an aspect of the present example embodiment, the measurement unit measures the time for one stride by using the time-series data of the vertical direction acceleration received from the first measurement device. The measurement unit measures, as the time for one stride, a time difference from a time relevant to the heel strike in the preceding gait cycle to a time relevant to the next heel strike for each of the left and right feet. According to the present aspect, by measuring the time for one stride by using the vertical direction acceleration data measured by the first measurement device, it is possible to accurately grasp the rhythm and speed of walking. In addition, according to the present aspect, accuracy of one-stride determination can be improved as compared with one-stride determination by the detection of the peaks of plantarflexion and dorsiflexion detected by the second measurement device and the third measurement device arranged on the left and right feet.

In an aspect of the present example embodiment, the information generation unit evaluates non-uniformity of the gait ability of the user by using the time-series data of the vertical direction acceleration received from the first measurement device. The information generation unit generates information according to the evaluation result of the non-uniformity of the gait ability of the user. According to the present aspect, by evaluating the non-uniformity of the gait ability by using the time-series data of the vertical direction acceleration measured by the first measurement device, it is possible to specify a problem of the walking balance of the user and provide information for improvement.

In an aspect of the present example embodiment, the measurement unit measures the gait parameter by using sensor data received from at least one of the first measurement device, the second measurement device, or the third measurement device. The measurement unit can accurately measure the gait parameter related to the time by synchronizing pieces of the sensor data measured by the second measurement device and the third measurement device arranged on the left and right feet of the user with each other. The information generation unit acquires the evaluation result regarding the body of the user by using the machine learning model trained to output the evaluation result regarding the body according to the input of the gait parameter. The information generation unit generates gait information including action recommendation information that is optimized in accordance with the evaluation result and prompts the user to make a decision. According to the present aspect, by acquiring the evaluation result regarding the body from the gait parameter by using the machine learning model, it is possible to generate the optimal action recommendation information according to the individual situation of the user.

### Second Example Embodiment

Next, a gait analysis device in a second example embodiment will be described with reference to the drawings. The gait analysis device in the present example embodiment has a configuration in which the gait analysis device in the first example embodiment is simplified. For example, functions of components included in the gait analysis device in the present example embodiment are implemented by the functions of the components included in the gait analysis device according to the first example embodiment.

### Configuration

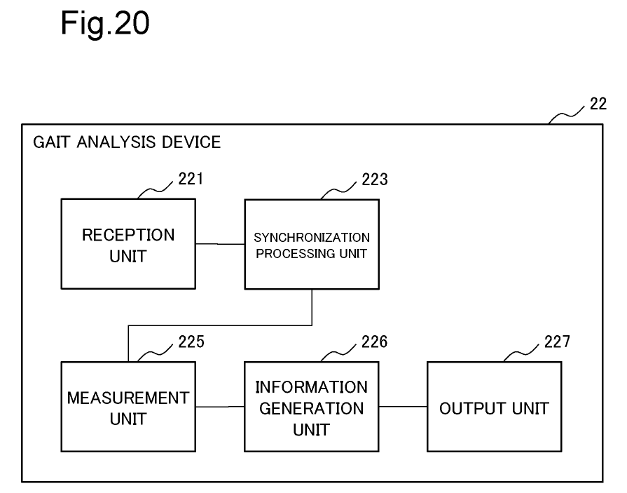

FIG. 20 is a block diagram illustrating an example of a configuration of the gait analysis device in the present disclosure. A gait analysis device 22 includes a reception unit 221, a synchronization processing unit 223, a measurement unit 225, an information generation unit 226, and an output unit 227.

The reception unit 221 receives pieces of sensor data measured by the first measurement device arranged along the body axis of the user, the second measurement device arranged on the left foot of the user, and the third measurement device arranged on the right foot of the user. The synchronization processing unit 223 synchronizes the pieces of the sensor data respectively received from the first measurement device, the second measurement device, and the third measurement device with each other. The measurement unit 225 measures a double support time by using the sensor data received from the first measurement device. The information generation unit 226 generates gait information according to the measured double support time. The output unit 227 outputs display information including the generated gait information.

### Operation

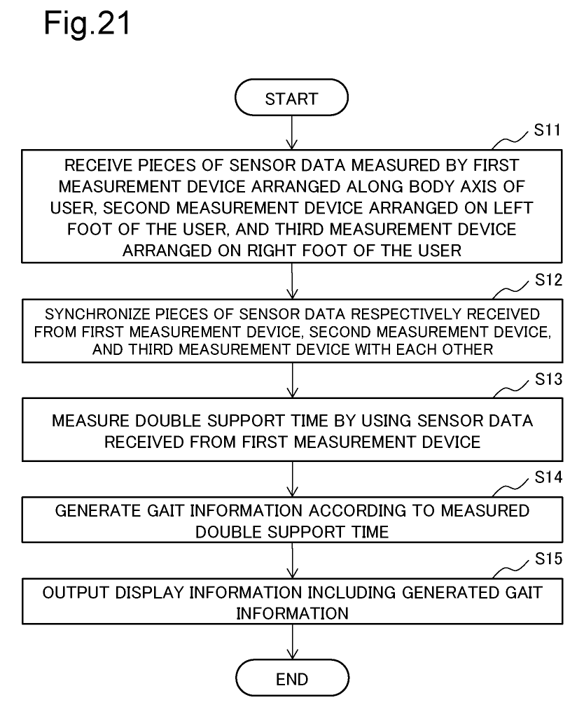

FIG. 21 is a flowchart illustrating an example of an operation of the gait analysis device in the present disclosure. In the description of the processing as per the flowchart in FIG. 21, a component of the gait analysis device 22 is assumed as an operating subject. The operating subject of the processing as per the flowchart in FIG. 21 may be the gait analysis device 22.

In FIG. 21, first, the reception unit 221 receives pieces of sensor data measured by the first measurement device arranged along the body axis of the user, the second measurement device arranged on the left foot of the user, and the third measurement device arranged on the right foot of the user (step S21).

Next, the synchronization processing unit 223 synchronizes the pieces of the sensor data respectively received from the first measurement device, the second measurement device, and the third measurement device with each other (step S22).

Next, the measurement unit 225 measures a double support time by using the sensor data received from the first measurement device (step S23).

Next, the information generation unit 226 generates gait information according to the measured double support time (step S24).

Next, the output unit 227 outputs display information including the generated gait information (step S25).

In the case of using sensor data measured according to the movement of the left and right feet, it is necessary to detect an inflection point of time-series data of the sensor data. For the person with weak gait, since the curvature of the time-series data of the sensor data decreases and the sampling rate is low, the inflection point of the time-series data of the sensor data cannot be accurately detected. In the present example embodiment, the double support time is measured by use of the sensor data measured by the first measurement device arranged along the body axis of the user without use of the sensor data measured according to the movement of the left and right feet. For that reason, according to the present example embodiment, the double support time can be accurately measured by use of the sensor data measured by the first measurement device arranged along the body axis of the user.

### Hardware

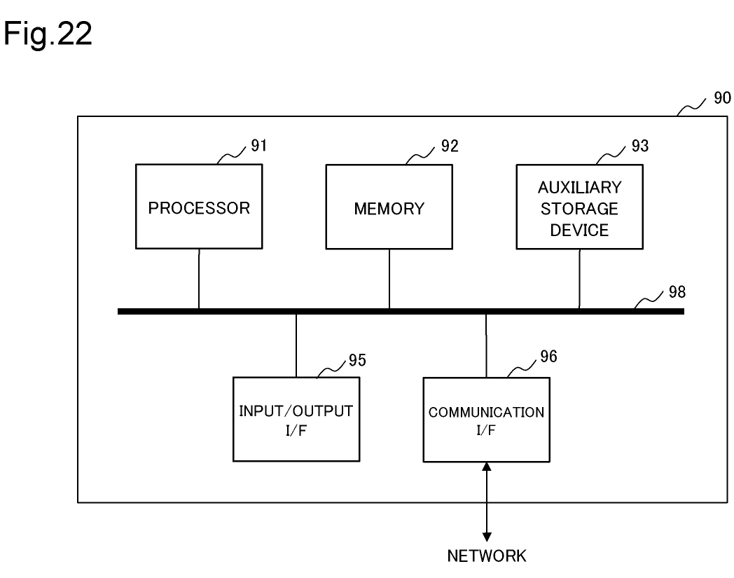

Next, a hardware configuration that executes control and processing in the present disclosure will be described with reference to the drawings. FIG. 22 is a block diagram illustrating an example of a hardware configuration that executes control and processing in the present disclosure. Here, an information processing device 90 (computer) is illustrated as an example of the hardware configuration. The information processing device of FIG. 22 is a configuration example for executing the control and processing in the present disclosure, and does not limit the scope of the present disclosure.

As illustrated in FIG. 22, the information processing device 90 includes a processor 91, a memory 92, an auxiliary storage device 93, an input/output interface 95, and a communication interface 96. In FIG. 22, the interface is abbreviated as an I/F. The information processing device 90 may include a plurality of pieces of at least one of the processor 91, the memory 92, the auxiliary storage device 93, the input/output interface 95, or the communication interface 96. The processor 91, the memory 92, the auxiliary storage device 93, the input/output interface 95, and the communication interface 96 are connected to each other via a bus 98 in such a way that data communication is allowed. The processor 91, the memory 92, the auxiliary storage device 93, and the input/output interface 95 are connected to a network such as the Internet or an intranet via the communication interface 96.

The processor 91 loads a program (command) stored in the auxiliary storage device 93 or the like into the memory 92. For example, the program is a software program for executing the control and processing in the present disclosure. The processor 91 executes the program loaded into the memory 92. The processor 91 executes the control and processing in the present disclosure by executing the program. The processor 91 may be constituted by a single piece of hardware or may be constituted by a plurality of pieces of hardware.

The memory 92 is a storage device having an area into which a program is loaded. A program stored in the auxiliary storage device 93 or the like is loaded into the memory 92 by the processor 91. The memory 92 is implemented by, for example, a volatile memory such as a Dynamic Random Access Memory (DRAM). A nonvolatile memory such as a Magnetoresistive Random Access Memory (MRAM) may be applied as the memory 92. The memory 92 may be constituted by a single piece of hardware or may be constituted by a plurality of pieces of hardware.

The auxiliary storage device 93 stores various types of data such as programs. For example, the auxiliary storage device 93 is implemented by a local disk such as a hard disk or a flash memory. The auxiliary storage device 93 may be constituted by a single piece of hardware or may be constituted by a plurality of pieces of hardware. The auxiliary storage device 93 may be configured as external hardware. The memory 92 may be formed to store various types of data in such a way that the auxiliary storage device 93 can be omitted.

The input/output interface 95 is an interface for connecting the information processing device 90 and a peripheral device to each other in accordance with a standard or a specification. The communication interface 96 is an interface for connecting to an external system or device through a network such as the Internet or an intranet in accordance with a standard or a specification. The input/output interface 95 may be constituted by a single piece of hardware or may be constituted by a plurality of pieces of hardware. The input/output interface 95 and the communication interface 96 may be merged as an interface connected to an external device.

Input devices such as a keyboard, a mouse, and a touch panel may be connected to the information processing device 90, as necessary. Those input devices are used to input information and settings. In a case where the touch panel is used as the input device, a screen having a touch panel function serves as an interface. The processor 91 and the input device are connected to each other via the input/output interface 95.

The information processing device 90 may be provided with a display device for displaying information. In a case where the display device is provided, the information processing device 90 includes a display control device (not illustrated) for controlling display on the display device. The information processing device 90 and the display device are connected to each other via the input/output interface 95.

The information processing device 90 may be provided with a drive device. The drive device mediates reading of data and a program stored in a recording medium and writing of a processing result by the information processing device 90 to the recording medium between the processor 91 and the recording medium (program recording medium). The information processing device 90 and the drive device are connected to each other via the input/output interface 95.

The above is an example of the hardware configuration for enabling the control and processing in the present disclosure. The hardware configuration of FIG. 22 is an example of the hardware configuration for executing the control and processing in the present disclosure, and does not limit the scope of the present disclosure. A program for causing a computer to execute the control and processing in the present disclosure is also included in the scope of the present disclosure.

A program recording medium in which a program for executing processing in the present example embodiment is recorded is also included in the scope of the present invention. For example, the program recording medium is a non-transitory computer-readable recording medium. The recording medium can be implemented by, for example, an optical recording medium such as a Compact Disc (CD) or a Digital Versatile Disc (DVD). The recording medium may be implemented by a semiconductor recording medium such as a Universal Serial Bus (USB) memory or a Secure Digital (SD) card. The recording medium may be implemented by a magnetic recording medium such as a flexible disk, or other recording media.

The components in the present disclosure may be arbitrarily combined with each other. The components in the present disclosure may be implemented by software. The components in the present disclosure may be implemented by a circuit.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each example embodiment can be appropriately combined with other example embodiments.

Some or all of the example embodiments described above may also be described as, but are not limited to, the following supplementary notes. In the following supplementary notes, dependent items in each category may also depend on other categories. The description included in the following supplementary notes has significance as a basis for amendment.

### Supplementary note 1

A gait analysis device including

a reception unit that receives pieces of sensor data measured by a first measurement device arranged along a body axis of a user, a second measurement device arranged on a left foot of the user, and a third measurement device arranged on a right foot of the user,

a synchronization processing unit that synchronizes the pieces of the sensor data respectively received from the first measurement device, the second measurement device, and the third measurement device with each other,

a measurement unit that measures a double support time by using the sensor data received from the first measurement device,

an information generation unit that generates gait information according to the double support time measured, and

an output unit that outputs display information including the gait information generated.

### Supplementary note 2

The gait analysis device according to supplementary note 1, in which

the measurement unit

detects a peak of a vertical direction acceleration received from the first measurement device,

specifies the left and right feet to which the peak of the vertical direction acceleration is relevant, depending on whether an angular velocity in a horizontal plane at a time of an extreme value of the peak of the vertical direction acceleration detected is positive or negative, and

measures, as the double support time, a time difference between a time of a heel strike of one foot and a time of a toe off of another foot in an identical gait cycle, for each of the left foot and the right foot specified.

### Supplementary note 3

The gait analysis device according to supplementary note 2, in which

the measurement unit,

by using time-series data of the vertical direction acceleration received from the first measurement device,

measures a time difference from a time relevant to a heel strike of the left foot to a time relevant to a toe off of the right foot as the double support time in a state where the left foot is stepped forward, and

measures a time difference from a time relevant to a heel strike of the right foot to a time relevant to a toe off of the left foot as the double support time in a state where the right foot is stepped forward.

### Supplementary note 4

The gait analysis device according to supplementary note 1, in which

the measurement unit

measures a time difference from a time relevant to a heel strike to a time relevant to a toe strike as a load time, for each of the left and right feet, by using time-series data of a vertical direction acceleration received from the first measurement device.

### Supplementary note 5

The gait analysis device according to supplementary note 1, in which

the measurement unit

measures a time difference from a time relevant to a heel strike in a preceding gait cycle to a time relevant to a next heel strike as a time for one stride, for each of the left and right feet, by using time-series data of a vertical direction acceleration received from the first measurement device.

### Supplementary note 6

The gait analysis device according to supplementary note 1, in which

the information generation unit

evaluates non-uniformity of gait ability of the user by using time-series data of a vertical direction acceleration received from the first measurement device, and

generates information according to an evaluation result of the non-uniformity of the gait ability of the user.

### Supplementary note 7

The gait analysis device according to supplementary note 1, in which

the measurement unit

measures a gait parameter by using the sensor data received from at least one of the first measurement device, the second measurement device, or the third measurement device, and

the information generation unit

acquires an evaluation result regarding a body of the user by using a machine learning model trained to output an evaluation result regarding a body according to an input of the gait parameter, and

generates the gait information including action recommendation information that is optimized in accordance with the evaluation result and prompts the user to make a decision.

### Supplementary note 8

A gait analysis system including

the gait analysis device according to any one of supplementary notes 1 to 7,

a first measurement device arranged along a body axis of a user,

a second measurement device arranged in association with a left foot of the user, and

a third measurement device arranged in association with a right foot of the user.

### Supplementary note 9

A gait analysis method including,

by a computer,

receiving pieces of sensor data measured by a second measurement device arranged on a left foot of a user, a third measurement device arranged on a right foot of the user, and a first measurement device arranged along a body axis of the user,

synchronizing the pieces of the sensor data respectively received from the first measurement device, the second measurement device, and the third measurement device with each other,

measuring a double support time by using the sensor data received from the first measurement device,

generating gait information according to the double support time measured, and

outputting display information including the gait information generated.

### Supplementary note 10

A program for causing a computer to execute

processing of receiving pieces of sensor data measured by a second measurement device arranged on a left foot of a user, a third measurement device arranged on a right foot of the user, and a first measurement device arranged along a body axis of the user,

processing of synchronizing the pieces of the sensor data respectively received from the first measurement device, the second measurement device, and the third measurement device with each other,

processing of measuring a double support time by using the sensor data received from the first measurement device,

processing of generating gait information according to the double support time measured, and

processing of outputting display information including the gait information generated.

Some or all of the configurations described in supplementary notes 2 to 8 dependent on the above-described supplementary note 1 can also be dependent on supplementary notes 9 and 10 by the same dependency relationship as in supplementary notes 2 to 8. Some or all of the configurations described as supplementary notes can be similarly dependent on not only supplementary notes 1, 9, and 10, but also diverse pieces of hardware and software, various recording means for recording software, or systems without departing from the above-described example embodiments.

1. A gait analysis device to measure a double support time using sensor data measured by a plurality of measurement devices, the gait analysis device comprising:
a memory storing instructions; and
a processor connected to the memory and configured to execute the instructions to:
receive, via wireless communication, pieces of sensor data measured by a first measurement device arranged along a body axis of a user, a second measurement device arranged on a left foot of the user, and a third measurement device arranged on a right foot of the user;
synchronize the pieces of the sensor data respectively received from the first measurement device, the second measurement device, and the third measurement device with each other;
measure a double support time by using the sensor data received from the first measurement device;
generate gait information according to the double support time measured; and
transmit display information including the gait information generated to a mobile terminal used by the user for displaying the display information on a screen of the mobile terminal.

2. The gait analysis device according to claim 1, wherein
the processor is configured to execute the instructions to:
detect a peak of a vertical direction acceleration received from the first measurement device;
specify the left and right feet to which the peak of the vertical direction acceleration is relevant, depending on whether an angular velocity in a horizontal plane at a time of an extreme value of the peak of the vertical direction acceleration detected is positive or negative; and
measure, as the double support time, a time difference between a time of a heel strike of one foot and a time of a toe off of another foot in an identical gait cycle, for each of the left foot and the right foot specified.

3. The gait analysis device according to claim 2, wherein
the processor is configured to execute the instructions to:
by using time-series data of the vertical direction acceleration received from the first measurement device,
measure a time difference from a time relevant to a heel strike of the left foot to a time relevant to a toe off of the right foot as the double support time in a state where the left foot is stepped forward; and
measure a time difference from a time relevant to a heel strike of the right foot to a time relevant to a toe off of the left foot as the double support time in a state where the right foot is stepped forward.

4. The gait analysis device according to claim 1, wherein
the processor is configured to execute the instructions to
measure a time difference from a time relevant to a heel strike to a time relevant to a toe strike as a load time, for each of the left and right feet, by using time-series data of a vertical direction acceleration received from the first measurement device.

5. The gait analysis device according to claim 1, wherein
the processor is configured to execute the instructions to
measure a time difference from a time relevant to a heel strike in a preceding gait cycle to a time relevant to a next heel strike as a time for one stride, for each of the left and right feet, by using time-series data of a vertical direction acceleration received from the first measurement device.

6. The gait analysis device according to claim 1, wherein
the processor is configured to execute the instructions to:
evaluate non-uniformity of gait ability of the user by using time-series data of a vertical direction acceleration received from the first measurement device; and
generate information according to an evaluation result of the non-uniformity of the gait ability of the user.

7. The gait analysis device according to claim 1, wherein
the processor is configured to execute the instructions to:
measure a gait parameter by using the sensor data received from at least one of the first measurement device, the second measurement device, or the third measurement device;
acquire an evaluation result regarding a body of the user by using a machine learning model trained to output an evaluation result regarding a body according to an input of the gait parameter, and
generate the gait information including action recommendation information that is optimized in accordance with the evaluation result and prompts the user to make a decision.

8. A gait analysis system comprising:
the gait analysis device according to claim 1;
a first measurement device arranged along a body axis of a user;
a second measurement device arranged in association with a left foot of the user; and
a third measurement device arranged in association with a right foot of the user.

9. A gait analysis method executed by a computer to measure a double support time using sensor data measured by a plurality of measurement devices, the gait analysis method comprising:
receiving, via wireless communication, pieces of sensor data measured by a second measurement device arranged on a left foot of a user, a third measurement device arranged on a right foot of the user, and a first measurement device arranged along a body axis of the user;
synchronizing the pieces of the sensor data respectively received from the first measurement device, the second measurement device, and the third measurement device with each other;
measuring a double support time by using the sensor data received from the first measurement device;
generating gait information according to the double support time measured; and
transmitting display information including the gait information generated to a mobile terminal used by the user for displaying the display information on a screen of the mobile terminal.

10. A non-transitory recording medium with a program to measure a double support time using sensor data measured by a plurality of measurement devices, the program causing a computer to perform the following processes:
receiving, via wireless communication, pieces of sensor data measured by a second measurement device arranged on a left foot of a user, a third measurement device arranged on a right foot of the user, and a first measurement device arranged along a body axis of the user;
synchronizing the pieces of the sensor data respectively received from the first measurement device, the second measurement device, and the third measurement device with each other;
measuring a double support time by using the sensor data received from the first measurement device;
generating gait information according to the double support time measured; and
transmitting display information including the gait information generated to a mobile terminal used by the user for displaying the display information on a screen of the mobile terminal.
